(19) United States
(12) Patent Application Publication
(10) Pub. No.: US 2021/0077514 A1
(43) Pub. Date: Mar. 18, 2021

(54) Nutraceutical Composition for Improving Overall Health

(71) Applicant: Gautam Umesh Vora, Mumbai (IN)

(72) Inventor: Gautam Umesh Vora, Mumbai (IN)

(21) Appl. No.: 16/688,312

(22) Filed: Nov. 19, 2019

(30) Foreign Application Priority Data
Sep. 16, 2019 (IN) 201921037273

(51) Int. Cl. A61K 31/706; A61K 31/4375; A61K 31/05; A61K 31/132; A61K 31/09; A61K 31/122; A23L 33/10

(52) U.S. Cl. CPC A61K 31/706; A61K 31/4375; A61K 31/05; A23L 33/10; A61K 31/09; A61K 31/122; A61K 31/132

(57) ABSTRACT

A nutraceutical composition is provided. The nutraceutical composition includes nicotinamide mononucleotide and one or more compounds selected from a group consisting astaxanthin, berberine, pterstobline, resveratrol, metformin, wherein proportion of the nutraceutical composition comprises 15.28 to 66.67% w/w nicotinamide mononucleotide; 0.23 to 3.33% w/w astaxanthin; 50 to 61.35% w/w berberine; 7.12 to 33.33% w/w pterstobline; 23.47 to 81.45% w/w resveratrol; and 34.43 to 56.98% w/w metformin. The present disclosure provides various advantages, including but not limited to, efficiency with respect to cell absorption and synergistic effect in terms of therapeutic efficacy.

This Application claims priority from a complete patent application filed in India having Patent Application No. 201921037273, filed on Sep. 16, 2019 and titled “A NUTRACEUTICAL COMPOSITION FOR IMPROVING OVERALL HEALTH”

### FIELD OF THE INVENTION

Embodiments of the present disclosure relate to nutraceutical composition, and more particularly to, a nutraceutical composition for improving overall heath.

### BACKGROUND

Over the past few decades, human health in general has been noticed to be deteriorating with every new generation. People, now, often face multiple heath issues, such as but not limited to, obesity, diabetes, low endurance, blood pressure, and cholesterol.

The aforementioned issues may be due to, including but not limited to, sedentary lifestyle, less or no exercise, diet habits, varied sleep patterns, stress, working for long hours and the like. Today, people are constantly working long hours, leading to less or no time to maintain a healthy lifestyle, which then leads to numerous health issues.

One health issue may lead to another and so on, until the human body fails, if the health issues are not addressed at an early stage. In few cases, such as, including but not limited to, cancer, an individual may not realise until the body is severely affected, which may lead to deleterious consequences.

In few cases, there would be number of pills or tablets to be consumed for every health issue diagnosed. Too many pills to be consumed may a cause side effects in the patient or the patient may miss out regular intake of pills.

Sometimes, in order to prevent health related issues, an individual may be prescribed pills, such as, including but not limited to, vitamin pills, omega-3 pills, iron, calcium, potassium and the likes. Usually, not every issue or disease can be prevented and sometimes, one pill may not contain components to prevent one or more issues or diseases.

Therefore, there is a need for a nutraceutical composition which can overcome the aforementioned issues.

### BRIEF DESCRIPTION

In accordance with one embodiment of the disclosure, a nutraceutical composition is provided. The nutraceutical composition includes nicotinamide mononucleotide and one or more compounds selected from a group consisting astaxanthin, berberine, pterstobline, resveratrol, metformin, wherein proportion of the nutraceutical composition comprises 15.28 to 66.67% w/w nicotinamide mononucleotide; 023 to 3.33% w/w astaxanthin; 50 to 61.35% w/w berberine; 7.12 to 33.33% w/w pterstobline; 23.47 to 81.45% w/w resveratrol; and 34.43 to 56.98% w/w metformin.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope.

### DETAILED DESCRIPTION

For the purpose of promoting an understanding of the principles of the disclosure, reference will now be made to the embodiment illustrated in the specific language will be used to describe them. It will nevertheless be understood that no limitation of the scope of the disclosure is thereby intended. Such alterations and further modifications in the illustrated nutraceutical product, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a nutraceutical product that comprises a list of steps does not include only those components but may include other components not expressly listed or inherent to such a nutraceutical product. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs.

In the following specification and the claims, reference will be made to a number of terms, which shall be defined to have the following meanings. The singular forms “a”, “an”, and “the” include plural references unless the context clearly dictates otherwise.

The term “nutraceutical composition” shall mean the particular components and amounts of the components in the form of energy nutrients, which are employed in the present invention to meet the nutrient deficiency generated by the stimulation of the cells and the resultant increase in overall immunity.

Embodiments of the present disclosure relate to a nutraceutical composition for overall improvement of health.

The present disclosure for a nutraceutical composition which is 100% natural and/or organic anchor combination of both without use of any additives in the form of one or more artificial colorants, one or more artificial flavours, or any unhealthy one or more chemicals.

In one embodiment, the nutraceutical composition includes nicotinamide mononucleotide and at least one compound selected from a group consisting astaxanthin, berberine, pterstobline, resveratrol, metformin.

Further, the nutraceutical composition may or may not include delivery agent to deliver the nutraceutical composition. In one embodiment, one delivery agent which may include a source of monounsaturated fatty acids or derivatives thereof. Preferably, the source of monosaturated fatty acid is an edible oil. Most preferably the edible oil is virgin olive oil or extra virgin olive oil, wherein the nutraceutical composition may be mixed with the virgin olive oil or extra virgin olive oil for improving delivery and absorption. The mixing enables dissolution of one or more ingredients of nutraceutical composition in the virgin olive oil or extra virgin olive oil. In the present case, the virgin olive oil or extra virgin olive oil act as a vehicle for the delivery of bioactive compounds. In one such embodiment, the virgin olive oil or the extra virgin olive oil may be mixed in quantity of 0.1 to 3 ml to the nutraceutical composition.

The composition may be formulated for administration utilising a suitable route. The composition may be formulated for oral, topical, intravenous, intramuscular, intrarectal, transdermal, subcutaneous, sublingual or intranasal administration. An oral or topical route is preferred.

When an oral route is chose, the nutraceutical composition may be formulated as a food ingredient, food additive, tablet, capsulate including soft gelatine capsules, caplet, liquid including syrup and oil, suspension, powder, granules, chewing gum or the like.

When a topical route is chosen, the nutraceutical composition may be formulated as a liquid, oil, paste, solution, dispersion, emulsion, lotion, gel, varnish or cream.

Certain embodiments provide liquid dosage forms for oral administration, including pharmaceutically acceptable emulsions, solutions, suspensions, and syrups, which may contain other components including inert diluents; adjuvants such as wetting agents, emulsifying and suspending agents; and sweetening, and flavouring agents. Controlled release oral formulations may be provided.

Controlled release may include, but is not limited to, delayed release and pH-dependent release. In certain embodiments, the nicotinamide mononucleotide and pterostilbene, or derivatives thereof can be incorporated into microcapsules, micro particulates, nano particulates, etc. through use of coatings to affect release of the active principle. In certain embodiments, nicotinamide mononucleotide and pterostilbene, or derivatives thereof can be incorporated into an inert matrix which permits release by either diffusion or leaching mechanisms, e.g., gums. Slowly degenerating matrices may also be incorporated into the formulation. Modified release oral formulations may be provided.

Modified release may allow for specific release profiles. Extended release oral formulations may be provided. Extended release may allow for release of active ingredients over a desired time period.

Opacifiers are used to opacify the capsule shell when the encapsulated active agents are light sensitive. Suitable opacifiers include, but not limited to, titanium dioxide, zinc oxide, calcium carbonate and combinations thereof. In an embodiment, the opacifier is titanium dioxide.

Colorants can be used to for marketing and product identification and/or differentiation purposes. Suitable colorants include synthetic and natural dyes and combinations thereof.

Humectants can be used to suppress the water activity of the soft gel. Suitable humectants include glycerine and sorbitol, which are often components of the plasticizer composition. Due to the low water activity of dried, properly stored soft gels, the greatest risk from microorganisms comes from moulds and yeasts. For this reason, preservatives can be incorporated into the capsule shell. Suitable preservatives include alkyl esters of p-hydroxy benzoic acid such as methyl, ethyl, propyl, butyl and heptyl or combinations thereof.

As used herein, the term “nicotinamide mononucleotide” is defined as a nucleotide derived from nicotinamide. Like nicotinamide mononucleotide, NMN is a derivative of niacin, and humans have enzymes that can use NMN to generate nicotinamide adenine dinucleotide.

Nicotinamide mononucleotide helps with respect to slowing aging process, repairing of DNA, reduction of weight, endurance, neurological function, benefits for heart, energy, enhancement of vision, metabolic syndrome and diabetes and Alzheimer's disease.

According to the study of Mills et al. (2016, pp. 795-806) on mice, Nicotinamide Mononucleotide (NMN) was observed to reduce the age-related physiological declines within them. By treating old mice with NMN, all biochemical aspects related to the process of ageing was reversed within them. Increasing the levels of NAD+ within the old mice also helped to restore the mitochondrial function within them to the level which is found in young mice. NMN also helped to restore the homeostasis of mitochondria and important biochemical markers of the muscle-health within those mice that were 22 months old to the level which was similar to those mice who were six months old.

Another study by Li et al. (2017, pp. 1312-1317) indicated that supplementation with Nicotinamide Mononucleotide could help to repair the DNA within the cells, which gets damaged from radiation. After one week's treatment with NMN supplement, it was discovered that the cells within the aged mice were indistinguishable from the cells of the young mice.

In one study, the supplement of Nicotinamide Mononucleotide was converted to NAD+ and immediately used within fifteen minutes, which resulted in significant rises within the levels of NAD+ over sixty minutes. It was found in the study that administering Nicotinamide Mononucleotide, which is considered as an essential intermediate of NAD+, can act as a beneficial intervention in treating pathophysiology of age and diet T2D. One surprising result which was discovered in this study was related to the dosage of NMN. It was found that only one dose of Nicotinamide Mononucleotide was sufficient in normalizing the impaired tolerance of the glucose (Yoshino, Mills, Yoon, & Imai 2011, pp. 528-536).

Another study by Uddin, Youngson, Sinclair and Morris (2016, p. 258) on Nicotinamide Mononucleotide indicated that the levels of NAD (+) were significantly increased by it within both liver and muscle. The supplementation of Nicotinamide Mononucleotide was also observed to induce the same reversal of the intolerance of glucose. In addition to this, it was also discovered that enhanced metabolism of fat within the liver because of post weaning supplementation of Nicotinamide Mononucleotide, as well as exercise were highly important contributors in dealing with the problem of adiposity.

The study of Wei et al. (2017, p. 717) indicated that the treatment of Nicotinamide Mononucleotide was also found to lower the death rate of brain cell, as well as oxidative stress. These findings further helped to support the neuroprotection of NAD+/NMN. Another study of Long et at (2015, p. 19) discovered that NMN helped to restore the function of mitochondrial respiratory effectively. Similarly, it was also discovered by Wang et al. (2016, pp. 1-9) that NMN has the ability to restore cognition within AD-model mice. Likewise, the research of Yao, Yang, Gao and Jia (2017, pp. 133-140) revealed that treatment of NMN has the potential to prevent cognitive impairments.

According to the research of Yamamoto et al. (2014), it was found that Nicotinamide Mononucleotide significantly helped to increase the NAD+ levels within the heart. Moreover, it also helped to protect the heart from injuries of I/R. Furthermore, the study of De Picciotto et al. (2016, pp. 522-530) highlighted that NMN was highly effective in lowering vascular-oxidative stress. It's treatment also aided in normalizing the aortic stiffness that was observed within aged mice. The findings also suggested that NMN can be used as a novel strategy to combat the arterial ageing process. Similarly, Zhang et al. (2017, pp. 64-73) concluded in their research that NMN could lower the myocardial inflammation by cutting off the initial signal of inflammation.

In the research of Martin et al. (2017) in which Nicotinamide Mononucleotide was administered on FXN-KO mouse, it was discovered that the cardiac function of that mouse was restored near the normal levels after NMN's administration. The supplementation of NMN also helped to restore the metabolism of the energy. Similarly, it also significantly reduced the energy expenditure of the whole body, as well as the wasting of cardiac energy.

The study of Lin et al. (2016, pp. 69-85) highlighted that the exogenous Nicotinamide Mononucleotide can help to prevent degeneration of photoreceptor and can aid in restoring vision. Moreover, it can also help to prevent retinal dysfunction in those degenerations that are induced by light.

Within the beta cells of the pancreas, secretion of the insulin is regulated by the NAD+ in response to the dietary ingestion of the glucose (Canto et al. 2010, pp. 213-219), Moreover, the activity of the insulin and homeostasis of the glucose are also governed by the NAD+ within the tissue of the adipose, liver and skeletal muscle (Fulco et al. 2008, pp. 661-673). Studies have indicated that usual biosynthesis of NAD+ can be drastically compromised within diabetic patients that can lead to intolerance of glucose and insulin resistance. It is observed that within skeletal muscle, liver and tissue of adipose, if the levels of NAD+ are restored, then Nicotinamide Mononucleotide can enhance the sensitivity of the insulin and can aid in reversing diabetes' course.

Dyslipidaemia can also be corrected by NMN, which is referred to as the condition where triglycerides and cholesterol are increased within the bloodstream (Caton et al. 2011, pp. 3083-3092). Insulin resistance and Dyslipidaemia are considered as an important characteristic of metabolic syndrome, which is a disorder that has affected around 35% of adults in the United States and has significantly raised the chances of strokes, heart disease and diabetes within them. In addition to this, NMN has also been observed to normalise the genes' expression that oversees the circadian rhythms. Moreover, as stated above, it also reduces inflammation and oxidative stress that are all significant underlying causes of metabolic syndrome, as well as type 2 diabetes. However, the interesting discoveries of the research that were conducted by Yoshino et al. (2011, pp. 528-536) highlighted that Nicotinamide Mononucleotide could play an important assistive role, in conjunction with lifestyle and dietary changes to treat and prevent metabolic syndrome and type 2 diabetes.

According to one report, Alzheimer's disease is increasing significantly, and around 5.7 million people in America are living with this disease. It is expected that this number will rise to 14 million in the year 2050. Lifestyle and dietary changes are essential for slowing down the progression or preventing Alzheimer's disease altogether. It is observed that Nicotinamide Mononucleotide can aid to strengthen the beneficial impacts associated with lifestyle and dietary changes to prevent Alzheimer's disease by protecting the brain's health. Nicotinamide Mononucleotide prevents the development of amyloid-beta, which is an abnormal protein which is characterized by the adverse changes in the brain due to Alzheimer's disease. Moreover, NMN also reduces inflammation of the brain and synaptic losses. These effects of NMN slows down the degeneration process within the brain and can even aid to reverse the progression or growth of Alzheimer's disease (Yao, Yang, Gao, & Jia 2017, pp. 133-140).

As used herein, the term “astaxanthin” is defined as a carotenoid pigment that occurs in trout, microalgae, yeast, and shrimp, among other sea creatures. It's most commonly found in Pacific salmon and is what gives the fish its pinkish colour. An antioxidant, astaxanthin is said to have many health benefits.

Astaxanthin helps with respect to anti-aging, antioxidant effects, anti-lipid peroxidation activity, anti-inflammation, anti-diabetic activity, cardiovascular disease prevention, anticancer activity and immuno-modulation.

According to Kumi Tominaga et al, (2017, pp. 33-39), it was reported that long-term treatment with astaxanthin prevented the accumulation of age-related oxidative stress and inflammatory response in aging mice. Based on these collective results, oral astaxanthin supplements are expected to inhibit inflammation-mediated skin deterioration, such as wrinkle formation and skin moisture decline, which appears in aged skin. Thus, final conclusion was made that long-term astaxanthin supplementation may prophylactically inhibit skin deterioration induced over time by environmental damage and consequently retard the skin aging process via its anti-inflammatory effect.

According to a study by Ranga Rao Ambati et al. (2014, pp. 225-232). Astaxanthin contains a unique molecular structure which are responsible for the high antioxidant properties. Antioxidant activity of astaxanthin was 10 times more than zeaxanthin, lutein, canthaxanthin, β-carotene and 100 times higher than α-tocopherol. Antioxidant enzyme activities were evaluated in the serum after astaxanthin was supplemented in the diet of rabbits, showing enhanced activity of superoxide dismutase and thioredoxin reductase whereas paraoxonase was inhibited in the oxidative-induced rabbits. Antioxidant enzyme levels were increased when astaxanthin fed to ethanol-induced gastric ulcer rats.

Astaxanthin has a unique molecular structure which enables it to stay both in and outside the cell membrane. It gives better protection than β-carotene and Vitamin C which can be positioned inside the lipid bilayer. If serves as a safeguard against oxidative damage by various mechanisms, like quenching of singlet oxygen; scavenging of radicals to prevent chain reactions; preservation of membrane structure by inhibiting lipid peroxidation; enhancement of immune system function and regulation of gene expression. Astaxanthin and its esters showed 80% anti-lipid peroxidation activity in ethanol induced gastric ulcer rats and skin cancer rats. Astaxanthin inhibited lipid peroxidation in biological samples reported by various authors.

Astaxanthin is a potent antioxidant to terminate the induction of inflammation in biological systems. Astaxanthin acts against inflammation. Park et al. (2010, pp. 1-10) reported astaxanthin reduced the DNA oxidative damage biomarker inflammation, thus enhancing immune response in young healthy adult female human subjects. Haines et al. (2010, pp. 128-136) reported lowered bronchoalveolar lavage fluid inflammatory cell numbers, and enhanced cAMP, cGMP levels in lung tissues after feeding astaxanthin with Ginkgo biloba extract and Vitamin C. Another study showed astaxanthin esters and total carotenoids from Haematococcus exerted a dose-dependent gastroprotective effect on acute, gastric lesions in ethanol-induced gastric ulcers in rats. Astaxanthin showed protective effect on high glucose induced oxidative stress, inflammation and apoptosis in proximal tubular epithelial cells. Astaxanthin is a promising molecule for the treatment of ocular inflammation in eyes as reported by the Japanese researchers. Astaxanthin can prevent skin thickening and reduce collagen reduction against UV induced skin damage.

Generally, oxidative stress levels are very high in diabetes mellitus patients, induced by hyperglycaemia, due to the dysfunction of pancreatic β-cells and tissue damage in patients. Astaxanthin could reduce the oxidative stress caused by hyperglycaemia in pancreatic β-cells and also improve glucose and serum insulin levels. Astaxanthin can protect pancreatic β-cells against glucose toxicity. It was also shown to be a good immunological agent in the recovery of lymphocyte dysfunctions associated with diabetic rats. In another study, ameliorate oxidative stress in streptozotocin-diabetes rats were inhibited by the combination of astaxanthin with α-tocopherol. Improved insulin sensitivity in both spontaneously hypertensive corpulent rats and mice on high fat plus high fructose diets was observed after feeding with astaxanthin in a study by Bhuvaneswari S et al. (2010, pp. 1406-1414). The urinary albumin level in astaxanthin treated diabetic mice was significantly lower than the control group. Some of the studies demonstrated that astaxanthin prevents diabetic nephropathy by reduction of the oxidative stress and renal cell damage.

According to Fassen R. G. et al. (2011., pp. 447-465), Astaxanthin is a potential therapeutic agent against atherosclerotic cardiovascular disease. Mice ted 0.08% astaxanthin had higher heart mitochondrial membrane potential and contractility index compared to the control group as indicated by Nakao R. et al. (2010, pp. 2721-2725). Astaxanthin effects on paraoxonase, thioredoxin reductase activities, oxidative stress parameters and lipid profile in hypercholesterolemic rabbits were evaluated. Astaxanthin prevented the activities of those enzymes from hypercholesterolemia induced protein oxidation at the dosages of 100 mg and 500 mg/100 g.

According to Chew B. P. et al. (2004, pp. 134-257) Astaxanthin showed significant antitumor activity when compared to other carotenoids like canthaxanthin and β-carotene. It also inhibited the growth of fibrosarcoma, breast, and prostate cancer cells and embryonic fibroblasts according to Daubrawa F. et al. (2005, pp. 135-250). Astaxanthin inhibited cell death, cell proliferation and mammary tumours in chemically induced male/female rats and mice indicated in a study by Jyonouchi H. et al. (2010, pp. 59-65).

Antioxidants in particular astaxanthin offer protection against free radical damage to preserve immune-system defences. There are reports on astaxanthin and its effect on immunity in animals under laboratory conditions however clinical research is lacking in humans. According to Jyonouchi H. et al. (1991, pp. 93-105) Astaxanthin showed higher immuno-modulating effects in mouse model when compared to β-carotene. Astaxanthin produced immunoglobulins in human cells in a laboratory study. Eight week-supplementation of astaxanthin in humans resulted in increased blood levels of astaxanthin and improved activity of natural killer cells Which targeted and destroyed cells infected with viruses.

As used herein, the term “berberine” is defined as a quaternary ammonium salt from the protoberberine group of benzylisoquinoline alkaloids found in such plants as Berberis (e.g. Berberis vulgaris—barberry, Berberis aristata—tree turmeric, Mahonia aquifolium—Oregon-grape, Hydrastis canadensis—goldenseal, Xanthorhiza simplicissima—yellowroot, Phellodendron amurense[2]—Amur cork tree, Coptis chinensis—Chinese goldthread, Tinospora cordifolia, Argemone mexicana—prickly poppy, and Eschscholzia califomica—Californian poppy). The berberine is usually found in the roots, rhizomes, stems, and bark

Berberine helps with respect to anti-aging, perimenopausal syndrome, cancer therapy, anti-diabetic, avoiding heart failure, antidepressant, weight loss, Alzheimer's disease and cholesterol metabolism.

According to Han X et al. (2016, pp. 235-248), the antioxidant activity of berberine has been widely demonstrated in aging cells in vitro and in aging models. Berberine has been found to decrease the level of constitutive DNA damage signalling. By using H2O2-induced senescence model, berberine significantly prevented the development of senescence via activation of AMPK pathway, which prevented hydrogen peroxide-induced impairment of the autophagic flux in senescent cells, and restored NAD+ levels in the senescent cells via a salvage pathway for NAD+ synthesis. In addition, berberine relieved oxidative stress in tissues in different organs, including liver, kidney, pancreas and central nervous system, providing multiple targets for the antioxidant effects of berberine in the systemic aging process, indicated by Li Z et al. (2014, pp. 71-80).

According to Cristiana Caliceti et al. (2015), there is growing evidence that Berberine can, at least partially, minimize the negative consequences on the organism caused by low oestrogens levels, without the unwanted side effects associated with commonly prescribed hormone replacement therapy (HRT). While the search for a HRT completely free of risks continues, BBR could represent a safe and efficient tool to sustain women during the menopausal transition.

According to Patil J B et al, (2010), accumulative evidences in both in vitro and in vivo studies using berberine demonstrated anti-cancer and anti-inflammatory properties in different cancer cells. Berberine inhibits the proliferation of MCF-7 breast cancer cells through a mitochondria and caspase dependent apoptotic pathway. It is possible that berberine may serve as a potential naturally occurring compound for breast cancer therapy. Another study by Tillhon M et al. (2012), addresses the properties and therapeutic use of berberine and concludes it to be a promising anticancer drug lead.

Berberine has been shown to regulate glucose and lipid metabolism in vitro and in vivo. In a pilot study by Jun Yin et al. (2008, pp. 712-717), berberine reduced serum cholesterol, triglycerides and LDL-C. Berberine is a potent oral hypoglycaemic agent with modest effect on lipid metabolism. It may serve as a new drug candidate in the treatment of type 2 diabetes.

BBR has been recognized as being capable of decreasing cardiovascular through reducing oxidative stress, low-density lipoprotein (LDL), triglycerides, and insulin resistance and improving the mood. In a clinical trial carried out on chronic heart failure patients, BBR decreased the frequency and complexity of ventricular premature complexes and increased the left ventricular ejection fraction, according to a study by Zeng X. H. et al. (2001, pp. 308-311).

Sigma receptors play an important role in the modulation of various neurotransmitters. Recent studies by Takebayashi M et al. (2004, pp. 208-213), have provided further evidence for the involvement of sigma receptors in the pathophysiology of major depression psychiatric disturbances. Berberine affects sigma receptor 1 similar to many antidepressant drugs, indicating its potential for the treatment of major depression.

Berberine has been shown to be a potential drug to treat obesity by downregulation of adipogenesis and lipogenesis. Mice treated with berberine were found to contain shrunk adipocytes, based on a study conducted by Zhang Z et al. (2014). Berberine exerts its long-term body weight losing effect. According to Brusq J M et al. (2006, pp. 1281-1288), Berberine was reported to inhibit cholesterol and triglyceride synthesis in HepG2 cells, a human hepatoma cell line, and primary hepatocytes. There is no direct evidence in humans showing the protective effect of berberine on NAFLD (non-alcoholic fat liver disease), but an indirect clinical investigation by Di Pierro F et al, (2012, pp. 213-217) suggests that berberine supplement may suppress NAFLD, as it reduces alanine and aspartate transaminase levels in patients with T2DM (type 2 diabetes mellitus).

There is substantial evidence from observational studies and clinical trials that conventional risk factors such as hypertension, diabetes, and dyslipidaetnia play a role in the development of Alzheimer's disease (AD). Targeting these risk factors will minimize the burden of AD in our aging population. Berberine can not only limit the role of these risk factors but also improve metabolic syndrome associated with AD as well. According to Zhiyou Cai et al. (2016, pp. 2509-2520), berberine may be a promising target to prevent and treat AD in the future on the basis of basic molecular biology research and clinical trials.

Cholesterol clearance in the liver is achieved through the conversion of cholesterol into bile acids and/or secreted as free cholesterol in bile. It is demonstrated that BBR increases cholesterol excretion from the liver into the bile and is eliminated via the faeces, indicated in a study by Li X. Y. et al. (2015, p. 13). According to another study by Guo Y. et al. (2016, p. 16), BBR supplementation alters bile acid profile by increasing primary bile acids while decreasing secondary bile acids in the liver and serum. BBR also activates cholesterol 7 alpha-hydroxylase expression and catalytic activity which also regulates bile acid synthesis. Taken all together, BBR enhances both cholesterol catabolism and bile acid excretion.

According to Zhiyou Cai et al. (2016, pp. 2509-2520), several therapeutic effects of berberine have been identified against cancer, obesity, congestive heart failure, inflammation, atherosclerosis, neurodegenerative diseases, rheumatoid arthritis, cardiovascular diseases, and metabolic disorders, such as dyslipidaemia, impaired fasting glucose, metabolic syndrome, and diabetes.

As used herein, the term “pterostilbene” is defined as a compound found in plants that is chemically similar to resveratrol but different in some important ways: Because of its ability to dissolve in body fat, PT lingers longer in the system and is absorbed better. PT works synergistically with NMN because it activates sirtuins.

Pterostilbene helps with respect to cancer, cardiovascular, haematology, heart health, Alzheimer's disease and diabetes management.

According to Rock C L et al. (2013, pp. 282-295) increasing rates of obesity and poor nutrition are major contributors to breast cancer occurrence in women. Several studies have shown that blueberry extract and pterostilbene inhibit breast cancer. Pterostilbene treatment of breast cancer cells has additionally been shown to alter cellular oxidative activity that may play an important role in pterostilbene-mediated cell death. Pterostilbene has been shown to exert anticancer effects in breast cancer through alteration of multiple cancer pathways both in vitro and in vivo. Studies performed by Chakraborty and colleagues found that pterostilbene reduced cell proliferation and induced apoptosis. The findings of Alosi, Mannal. and Chakraborty imply that pterostilbene alters cellular oxidation to facilitate mechanisms of apoptosis in breast cancer.

Several studies have shown that blueberries, and pterostilbene alike, exhibit protective effects against cardiovascular disease possibly due to induction of antioxidant enzymes. According to a study by Denise McCormack and David McFadden, Pterostilbene has demonstrated numerous protective benefits against atherosclerosis through regulation of vascular smooth muscle cells (VSMCs) and vascular endothelial cells (VECs).

Studies by Mikstacka R et al. (2010, pp, 57-63) have shown that reactive oxygen species (ROS)-induced haemolysis is a modifiable event that can be alleviated with antioxidant treatment. Specifically, treatments with blueberry extract and pterostilbene have been shown to protect RBCs (red blood cells) against ROS-induced haemolysis indicating a possible therapeutic effect in the treatment of haemolytic anaemia. It has been postulated that blueberries and its component pterostilbene protect RBCs against OS (oxygen species) by scavenging H2O2 (Hydrogen Peroxide), altering the harmful effects of ROS and increasing antioxidant activity.

Pterostilbene shows promise for protection against atherosclerosis, or hardening of the arteries, according to a 2012 study published in Apoptosis. In tests on human cells and on mice, scientists demonstrated that pterostilbene may inhibit the build-up of LDL cholesterol, or bad cholesterol. The study also found that pterostilbene may help fight oxidative stress, a destructive biological process thought to be a key factor in the development of heart disease.

Pterostilbene may help protect against Alzheimer's disease and aging-related cognitive decline, according to an animal-based study published in Neurobiology of Aging in 2012. In tests on mice, the study's authors determined that pterostilbene may help preserve cognitive function, in part by reducing inflammation.

In a study by Elango B et al. (2019, pp. 47-57), they investigated the antidiabetic role of pterostilbene (PTS) in streptozotocin (STZ)-induced diabetic model through Nuclear factor erythroid 2-related factor 2 (Nrf2)-mediated antioxidant mechanisms. The results clearly indicated that PTS maintains glucose homeostasis, suggesting the possibility that it is a future candidate for use in diabetes management.

In a study by Jose M. Estrela et al. (2013, pp. 65-78), the metabolism and pharmacokinetics of this stilbene in inflammatory dermatoses and photoprotection, cancer prevention and therapy, insulin sensitivity, blood glycemia and lipid levels, cardiovascular diseases, aging, and memory and cognition are addressed.

As used herein, the term “resveratrol” is defined as a type of natural phenol, and a phytoalexin produced by several plants in response to injury or, when the plant is under attack by pathogens such as bacteria or fungi. Sources of resveratrol in food include the skin of grapes, blueberries, raspberries, mulberries, and peanuts.

Resveratrol helps with respect to blood pressure level, anti-inflammatory, anti-aging, protection of brain, bile and gut microbiome production, balancing blood sugar level, combat obesity, potential to prevent cancer, potential to enhance bone health and heath of the liver.

According to Bonnefont-Rousselot (2016, p. 250) resveratrol has been considered to have the potential to reduce blood pressure level due to its antioxidant properties. Moreover, according to the conclusion of a review which was conducted in 2015, the increases doses of this supplement was observed to lower the pressure, which is exerted on the artery walls, whenever the heart beats. This type of pressure on the heart is referred to as systolic blood pressure (Liu et al. 2015, pp. 27-34). Within the readings of the blood pressure, it is observed to appear as the upper number. As the age of a person increases, it is noted that systolic blood pressure usually rises when due to the stiffness of the arteries. When it reaches a high level, then the risk for heart diseases in a person increases dramatically (López-Sepúlveda et al. 2008, pp. 1088-1095). However, it is observed that resveratrol can help to reduce this blood pressure level by aiding to create high amount nitric oxide that relaxes the blood vessels (Xia, Förstermann, Li 2014, pp. 16102-16121).

Apart from its antioxidant's indirect effects on the inflammation, it is observed that resveratrol can also influence the very specific processes related to inflammatory. For instance, it is noted that it helps to block the COX enzymes of inflammatory that are also considered as the primary target of typically utilized drugs related to anti-inflammation (NSAIDs, such as Motrin) and painkillers. However, as compared to these enzymes that are conventionally targeted, resveratrol's effects have been observed to run much deeper (Donnelly et al., 2004).

Resveratrol has also been considered to produce some beneficial effects on important pathways of diseases related to the process of ageing. According to cellular and animal research, resveratrol has the ability to combat with disorders related to age by imitating the impacts of caloric restriction that raises the lifespan of animals (Smoliga, Baur, & Hausenblas 2011, pp. 1129-1141). Similarly, as stated above, it also lowers inflammation on various levels (such as gene expression, immune cells, and enzymes) as well as reduces oxidative stress. Moreover, resveratrol also activates SIRTI that helps to turn off those genes that increase the ageing process (Xiong et al. 2015, pp. 1692-1701).

It is observed that within the brain, the glial cells help to protect and support the neuron. However, their dysfunction or damage is associated with several diseases in the brain, and any positive activity of them is dependent upon their ability to intake the amount of neurotransmitter glutamate. According to one cellular research, resveratrol can help to increase the uptake of glutamate within these cells, which in turn, helps to protect the brain from stroke and degeneration (Dos Santos et al. 2006, pp. 161-167). Similarly, within animal research, resveratrol has been observed to lower seizures and also protected the primary memory hub of the brain known as the hippocampus (Wu et al. 2009, p. 1393). Within the hippocampus, it also helps to raise IGF-1 that enhances cognition (Harada et al. 2011, pp. 1150-1159).

It is observed that arteries can also be protected against various diseases through gut microbiome. This is because, without APOE, it helps to protect blood vessels within mice, prevent dangerous bacteria from affecting their gut from secreting detrimental substances of oxidative (such as TMAO), and assists in balancing their microbiome. In addition to this, it also helps to increase beneficial or positive bacteria within the gut like Bifidobacterium and Lactobacillus that in turn increases bile acids' production (Chen et al., 2016).

According to the research of Timmers et al. (2011, pp. 612-622), resveratrol has been observed to aid in the metabolism of glucose. Within eleven obese but healthy men, the 150-milligram amount of resveratrol helped to increase insulin sensitivity within them, and after 30 days, their blood sugar levels were also decreased. Likewise, it also helps in raising the levels of PGC-1a and SIRT1. SIRT is considered as an enzyme which is highly essential to turn off the detrimental genes that increase the deposits of fat, inflammation, and blood sugars within the body. Whereas, healthy mitochondria are supported by PGC-1a.

Within cell-based researches, it has been observed that resveratrol helps to stop fat cells from producing new fats and triggers their death. It achieves this by shutting down the genes that increase weight gains like PPAR gamma (Rayalam et al. 2008, pp. 1367-1371). Moreover, it also activates those genes that help to improve mitochondrial health (UCP1, SIRT3) and energy utilization (Gerhart-Hines, pp. 1913-1923),

Numerous scientists and researchers are examining the effects of resveratrol to prevent the spreading, growth and development of cancer, which also includes metastatic cancer especially within the liver (Howells et al. 2011, pp. 1419-1425). In different cell-based researches, resveratrol has been found to protect the liver against cancer (Sun, Pan, Liu, & Wang 2002, p. 79). It is also observed to stop the growth rate of leukaemia cells, as well as the growth of other related cell types of cancer (Tsan, White, Maheshwari, & Chikkappa 2002, pp. 983-987).

There are various possibilities that are presented to the stem cells within the connective tissue, which may range from developing into muscle cells, joint cells, bone-building cells or fat cells. Resveratrol helps in activating the fat burning pathway (i.e., SIRT1) that helps in blocking those genes which increases storage of the fat (i.e., PPAR gamma). This sudden epigenetic change influences the stem cells to develop or change themselves into bone-building cells that can also enhance the health of the bone (Backesjö, Li, Lindgren, & Haldosén 2009, pp. 93-97). Moreover, by using vitamin K2 and Vitamin D, resveratrol can be synergized to increase mineralization of the bone, as well as to support their protection (Parazzini 2014, pp. 513-518).

It is also observed that resveratrol can help to protect the liver against various diseases and can also enhance the bile flow (Chen, Hu, Lu. & Shen 2015, pp. 402-407). Within animals, its effect helped to enhance and protect non-alcoholic diseases associated with fatty liver. Moreover, by blocking MMP-9 and MMP-2, it also helped to improve the conditions of the obstructed flow of bile (Pan, Lai, Tsai, & Ho 2014, pp. 147-171). Furthermore, resveratrol was also noted to protect liver of animals from serious damages that are caused by blood poisoning or sepsis that occur from severe infections (Xu et al. 2014, pp. 440-447). The below figures summarize some of the benefits and effects of resveratrol that have also been discussed within this report (Berman, Motechin, Wiesenfeld, & Holz 2017, p. 35).

As used herein, the term “metformin” is defined as the first-line medication for the treatment of type 2 diabetes. This is particular true in people who are overweight. It is also used in the treatment of polycystic ovary syndrome. Limited evidence suggests metformin may prevent the cardiovascular disease and cancer complications of diabetes.

Metformin is an FDA approved drug in common use in the US since the 1990s. It is the first-line drug of choice for prevention and treatment of type 2 diabetes (T2DM). The effect of metformin on aging has been extensively studied and has been associated with longevity in many rodent models. Metformin also extends the lifespan of nematodes, suggesting an evolutionarily conserved mechanism. A recent high impact study demonstrated that metformin reduces oxidative stress and inflammation and extends both lifespan and health span in a mouse model.

Notably, in the United Kingdom Prospective Diabetes Study (UKPDS) metformin, compared with other anti-diabetes drugs, demonstrated a decreased risk of cardiovascular disease. This has been suggested in other studies and meta-analyses and remains an active area of research.

Metformin helps with respect to extend lifespan and health span, anti-aging, Type2 diabetes, cardiovascular health, weight loss, inflammation, endothelial function and angiogenesis and psychological health,

There is an evidence that single gene mutations in nutrient-sensing pathways, such as insulin/insulin-like growth factor (IGF) signalling (Bartke et al., 2001) or the mechanistic target of rapamycin (mTOR) signalling pathways, extend lifespan and health span in invertebrates. These pathways have been evaluated in mammalian models, in which health span and lifespan have been extended by genetic manipulation or drugs (Johnson et al., 2013).

According to the research Metformin might be able to prevent aging in human cells by acting through the Nr12-GPx7 pathway. A study conducted by a team of researchers led by Professors Wang Chihchen and Liu Guanghui at the Institute of Biophysics of Chinese Academy of Sciences (CAS) found that chronic low-dose metformin treatment delays aging in human cells, specifically diploid fibroblasts and mesenchymal stem cells.

Previous research by the same group showed that a protein called endoplasmic reticulum-localized glutathione peroxidase 7 (GPx7) is a key enzyme involved in regulating protein folding and maintaining redox homeostasis. The researchers found that low-dose metformin treatment upregulates the expression of endoplasmic reticulum-localized GPx7 by activating a transcription factor called Nrf2.

The levels of GPx7 decrease as cells age and knocking down GPx7 accelerated the process of aging, the researchers said. Interestingly, the metformin-Nrf2-GPx7 axis is known to be involved in worm aging and the worm ortholog of human GPx7 is required for the positive effects of metformin on life span extension in worms.

This therapeutic profile of metformin supports its use for age-related diseases and longevity. Of significance, many studies have confirmed the positive effect of metformin on life span of worms, flies, mice, and rats.

Metformin enhances the activity of an enzyme found within our cells called adenosine monophosphate-activated protein kinase (AMPK). AMPK activation helps mimic the beneficial effects of calorie restriction, the best documented method of slowing and reversing biomarkers of human aging.

Metformin is a first-line pharmacological treatment for patients with type 2 diabetes mellitus because of its favourable overall profile, including its glucose-lowering ability, weight-neutral effects, and low risk of hypoglycaemia.

Clinical Trials: The Diabetes Prevention Program (DPP): Metformin improves glycaemic control includes the non-competitive inhibition of the mitochondrial glycerophosphate dehydrogenase enzyme, which alters hepatocellular redox state, thus reducing the conversion of lactate and glycerol to glucose, decreasing hepatic gluconeogenesis, The DPP was a randomized trial in U.S. adults at high risk for T2DM by virtue of obesity and impaired glucose tolerance (Knowler et al., 2002). Over 3,000 subjects were randomly assigned to placebo, metformin (850 mg twice daily), or a lifestyle-modification program. Metformin reduced the incidence of T2DM by 31% compared to placebo over a mean follow-up of ˜3 years and was effective in all age categories in preventing diabetes, defined by HbA1C level, including the ˜20% who were age 60 or older at baseline (Knowler et al., 2015). Further, metformin treatment was associated with improvement in cardiovascular disease (CVD) risk factors (Goldberg et al., 2013; Haffner et al., 2005) and subclinical atherosclerosis (coronary artery calcium) in male participants (Goldberg et al., 2015).

In age-dependent diseases, such as coronary artery disease, cerebrovascular disease, osteoporosis and Alzheimer's Disease, the pathogenesis appears to involve basic ageing processes, chronic damage from inflammation and dysregulated cellular metabolism. Mortality and morbidity in these diseases increase exponentially with advanced age. In contrast, age-related diseases have a temporal relationship with the age of the host but are not necessarily related to the ageing process. These diseases occur at a specific age, but with a further increase in age, they either decline in frequency or increase at a less than exponential rate. Examples are gout, multiple sclerosis and many (but not all) cancers.

As already mentioned, beyond the impact of metformin on glycaemic control, this drug is also proposed to alter mechanisms related to ageing. For example, inflammatory markers, such as interleukins and TNF, can activate a variety of cellular processes that lead to cellular and tissue damage. IL-6 can induce fibroblast proliferation and collagen production, leading to cardiac remodelling. It can also promote myocyte hypertrophy, depressed contractility and apoptosis. Metformin has been shown to alter inflammatory responses through suppression of NF-kB via AMP-activated protein kinase (AMPK)-dependent pathways. In addition, metformin reduces the production of ROS through reverse electron flux and via the mechanistic target of rapamycin (mTOR), leading to a reduction in superoxide, which may otherwise lead to DNA damage and mutations.

A recent cohort study of US older veterans with type 2 diabetes showed that metformin reduced CVD events among individuals with type 2 diabetes, according to the baseline risk. The likelihood of CVD was reduced by 6% among otherwise healthy individuals, by 18% among those at risk of frailty and by 48% among those at high cardiovascular risk. According to sources, metformin may also have cardioprotective effects (Eurich et al. 2013; Hong et al. 2013). Another recent large double-blind randomised, placebo-controlled trial evaluated the cardiometabolic effects of metformin in adults with type 1 diabetes (for ≥5 years) and high CVD risk. Participants had an average age of 55.2±8.5 years and 88% had overweight or obesity. After 3 years, there was no difference in the primary outcome of carotid artery intima-media thickness (a surrogate marker of CVD). Still, there were reductions in body weight, LDL-cholesterol, and also in atherosclerosis progression, based on maximal carotid artery intima-media thickness analysis. These findings highlight the potential of metformin for decreasing CVD risk.

Several studies have shown a small but beneficial level of weight loss with metformin therapy in individuals without diabetes, at risk, or with type 2 diabetes. Metformin is not approved as an anti-obesity medication, but practitioners often incorporate off-label use for individuals with obesity at high risk for diabetes.

Furthermore, metformin mildly reduces levels of high-sensitivity C-reactive protein, and improves endothelial function. While this may be partially related to weight loss, the additional impact of metformin on inflammation, endothelial function and angiogenesis, enhances the benefits of this drug against the ageing process. Notably, a pilot placebo-controlled study of women without diabetes showed that, besides improving variables of vascular function, metformin also improved measures taken during an exercise tolerance test: maximal ST-segment depression, Duke score and chest-pain incidence. These findings provide further evidence that metformin may reduce CVD, supporting its use as an additional therapy to reduce cardiovascular risk factors and complications, including death. Importantly, however, since this effect has been most widely reported in individuals with diabetes, further research is required to fully establish the impact of metformin on CVD risk in those without diabetes.

Depression: Regarding the psychological health, in a placebo-controlled Chinese study in participants with type 2 diabetes and mild to moderate depression, metformin improved depressive symptoms, possibly because of better glycaemic control. Considering the established relationship and high prevalence of depression in older individuals with or without diabetes, this outcome is highly relevant.

Cognitive function: There is also evidence that metformin alters cognition. Observational studies show reductions in mild cognitive impairment (MCI) and dementia among participants with diabetes taking metformin when compared with no medication or other glucose-lowering agents. For example, a Taiwanese study in individuals aged ≥50 years found that metformin use significantly decreased the risk of dementia compared with no medication (HR 0.76 [95% CI 0.58, 0.98]). In another study, researchers evaluated data from 365 individuals from the Singapore Longitudinal Aging Study, aged ≥55 years; they found that metformin use was associated with lower risk of MCI (OR 0.49 [CI 0.25, 0.95]). In another study in Taiwan, researchers analysed data from 67,731 individuals using an insurance database and found that dementia risk was lower in those taking metformin compared with other glucose-lowering medications. It must be noted that, because of their observational design, and despite adjusting models for confounders, such as age, education, diabetes duration, CVD and other risk factors, the possibility of residual confounding in these studies persists. Nonetheless, while these studies have limitations, a recent pilot clinical trial substantiated their findings by showing that metformin improved cognition in individuals without diabetes. Specifically, eighty individuals, aged 55 to 90 years, with amnestic MCI and without treated diabetes were randomly assigned to metformin or placebo and followed for 12 months. The participants treated with metformin showed improvements in the selective reminding test, even after adjusting for baseline values for the Alzheimer's Disease Assessment Scale-cognitive subscale (ADAS-cog) score.

On the other hand, concerns were recently raised that metformin may be associated with deleterious effects on cognitive function in older individuals. Researchers reported worsening tau aggregation and abnormal behaviour or impaired spatial memory and visual acuity in mouse models of ageing. Nevertheless, investigators from the DPPOS have just published an analysis of cognitive function, measured 8-10 years after therapy with metformin. While the results did not support benefit, they did not show negative impact from long-term metformin use.

In regard to the functional medical domain, as mentioned previously, the potential impact of metformin on skeletal muscle may be particularly helpful in sarcopenic obesity. It is yet to be established whether metformin enhances physical function and mobility or prevents their decline in older adults. However, in an observational study metformin seemed to decrease the likelihood of frailty and other age-related comorbidities.

Metformin has been successfully used for long-term treatment in older adults and it is the first-line therapy for type 2 diabetes. It has also been used long-term to prevent diabetes. For individuals who have never previously used metformin, metformin can be safely started at 500 mg per day and slowly titrate (to ensure tolerance) towards the target dose of 1000 mg twice daily, as long as GFR is above 45 ml min−1 [1,73 m]−2. Kidney function needs to be monitored in patients and there may be a need to stop metformin in those with signs of kidney disease (for example if there is an acute increase in serum creatinine). After the situation is resolved, metformin can he resumed with caution, as long as GFR remains above 30 ml min−1 [1.73 m]−2, lowering the dose to a maximum of 1000 mg per day if GFR stays above 30 but below 45 ml min−1 [1.73m]−2.

Regarding adverse events, there is ongoing debate whether or not metformin is associated with lactic acidosis. Nonetheless, the event frequency is so small that, in most clinical practices, the preventive approach is to temporally place metformin on hold in the setting of hospitalisation, acute kidney injury, use of iodinated-contrast procedures, or in the setting of acute severe illness with hypoxia (all of which increase the risk of lactic acidosis), following which metformin therapy is reimplemented and use is continued.

High concentrations of ceramides in the skeletal muscle are also proposed to be involved in the ageing process. This can lead to reduced myoblast proliferation, aberrant cell cycle regulation and a senescent myoblast phenotype. Cell studies showed that treatment with metformin can limit the negative effects of ceramides, thus potentially preventing myoblast senescence.

Increased healthcare costs in an ageing society, for which interventions to reduce the burden of NCDs and to promote healthy ageing are needed. Lifestyle modifications, including exercise interventions, have several advantages, but are difficult to adhere to and maintain over time. Metformin offers a cost-effective alternative that, besides controlling diabetes or reducing its risk, may improve mood and cognitive and physical functions. Two large metformin clinical trials in individuals without diabetes will soon start to evaluate the benefit of metformin treatment on these outcomes. The Veterans Affairs' Investigation of Metformin in Pre-Diabetes on Atherosclerotic Cardiovascular OuTcomes (VA-IMPACT; ClinicalTrials.gov registration no. NCT02915198) is a placebo-control study in individuals with CVD and intermediate hyperglycaemia, the latter defined as: one measure of glycated HbA1c 5.7-6.4% (38.8-46.4 mmol/mol); two measurements of fasting blood glucose (on separate days) between 5.6 mmol/l. and 6.9 mmol/l; or a 2-h blood glucose level between 7.8 mmol/l and 11.1 mmol/l following a 75 g glucose load OGTT; all in the absence of known diabetes or use of a glucose-lowering agent. The primary outcomes include the time to death from any cause, myocardial infarction, stroke, hospitalisation for unstable angina, or symptom-driven coronary revascularisation. The Targeting Ageing with Metformin (TAME) study is another major placebo-controlled trial in older adults without diabetes but at increased risk of functional decline. TAME will evaluate the potential ability of metformin to slow down the development of age-dependent and age-related diseases, including cancer, CVD and dementia. In addition, TAME will include outcomes of physical function and mobility. Both VA-IMPACT and TAME are in the final stages of approval and implementation, and aiming to begin recruitment this year, in 2017. Finally, the Early Prevention of Diabetes Complications in Europe (ePREDICE) is another large multicentre randomised clinical trial, already recruiting participants mostly in Europe, evaluating the impact of metformin (compared with a dipeptidyl peptidase-4 inhibitor) on microvascular complications and cognitive function in individuals with non-diabetic intermediate hyperglycaemia (impaired glucose tolerance, impaired fasting glucose, or both).

In one such embodiment, the proportion of the nutraceutical composition includes:
15.28 to 66.67% w/w nicotinamide mononucleotide,
0.23 to 3.33% w/w astaxanthin,
50 to 61.35% w/w berberine,
7.12 to 33.33% w/w pterstobline,
23.47 to 81.45% w/w resveratrol, and
34.43 to 56.98% w/w metformin.

In another embodiment, a specific proportion of the nutraceutical composition includes:
20.99% w/w nicotinamide mononucleotide
52.47% w/w resveratrol
0.31% w/w astaxanthin
26.23% w/w berberine

In yet another embodiment, a specific proportion of the nutraceutical composition includes:
39.76% w/w nicotinamide mononucleotide
9.94% w/w pterstobline
0.60% w/w astaxanthin
49.70% w/w berberine

In yet another embodiment, a specific proportion of the nutraceutical composition includes:
39.76% w/w nicotinamide mononucleotide
994% w/w pterstobline
0.60% w/w astaxanthin
49.70% w/w metformin

In yet another embodiment, a specific proportion of the nutraceutical composition includes:
44.15% w/w nicotinamide mononucleotide
55.19% w/w berberine
0.66% w/w astaxanthin

In yet another embodiment, a specific proportion of the nutraceutical composition includes:
28.45% w/w nicotinamide mononucleotide
71.12% w/w resveratrol
0.43% w/w astaxanthin

In yet another embodiment, a specific proportion of the nutraceutical composition includes:
44.15% w/w nicotinamide mononucleotide
0.66% w/w astaxanthin
55.19% w/w metformin

In yet another embodiment, a specific proportion of the nutraceutical composition includes:
79.05% w/w nicotinamide mononucleotide
19.76 w/w pterstobline
1.19% w/w astaxanthin

In yet another embodiment, a specific proportion of the nutraceutical composition includes:
80.00% w/w nicotinamide mononucleotid
20.00% w/w pterstobline

In yet another embodiment, a specific proportion of the nutraceutical composition includes:
44.44% w/w nicotinamide mononucleotide
55.56% w/w metformin

The nutraceutical composition may be administered as capsule, tablet, powder or drink.

The regular consumption of the nutraceutical composition completes the nutritional gaps in a person's diet and provides a complete and wholistic nutritional support. The person may feel improvement in following vitality and vigour, antioxidant activity, control of obesity/weight loss, depression, hypertension, diabetes, dyslipidaemia, Alzheimer's disease (AD) in aged, cholesterol metabolism, cancer, congestive heart failure, inflammation, atherosclerosis, neurodegenerative diseases, rheumatoid arthritis, low oestrogens levels, free radical damage to preserve immune-system defences, renal cell damage, cardiovascular diseases, and metabolic disorders, such as dyslipidaemia, impaired fasting glucose, metabolic syndrome etc.

The present disclosure provides various advantages, including but not limited to, efficiency with respect to cell absorption and synergistic effect in terms of therapeutic efficacy.

While specific language has been used to describe the disclosure, any limitations arising on account of the same are not intended. As would be apparent to a person skilled in the art, various working modifications may be made to the method in order to implement the inventive concept as taught herein.

The foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. The scope of embodiments is by no means limited by these specific examples.

I/we claim:

1. A nutraceutical composition comprising:
nicotinamide mononucleotide and at least one compound selected from a group consisting astaxanthin, berberine, pterstobline, resveratrol, metformin,
wherein proportion of the nutraceutical composition comprises
15.28 to 66.67% w/w nicotinamide mononucleotide;
0.23 to 3.33% w/w astaxanthin;
50 to 61.35% w/w berberine:
7.12 to 33.33% w/w pterstobline;
23.47 to 81.45% w/w resveratrol; and
34.43 to 56.98% w/w metformin.

2. The nutraceutical composition as claimed in claim 1, further comprises at least one delivery agent for delivering the nutraceutical composition, wherein the delivery agent is of 0.1 to 3 ml.

3. The nutraceutical composition as claimed in claim 1, wherein a specific proportion of the nutraceutical composition comprises:
44.15% w/w nicotinamide mononucleotide,
55.19% w/w berberine and
0.66% w/w astaxanthin.

4. The nutraceutical composition as claimed in claim 1, wherein a specific proportion of the nutraceutical composition comprises:
79.05% w/w nicotinamide mononucleotide,
19.76% w/w pterstobline and
1.19% w/w astaxanthin.

5. The nutraceutical composition as claimed in claim 1, wherein a specific proportion of the nutraceutical composition comprises:
28.45% w/w nicotinamide mononucleotide,
71.12% w/w resveratrol and
0.43% w/w astaxanthin.

6. The nutraceutical composition as claimed in claim 1, wherein a specific proportion of the nutraceutical composition comprises:
20.99% w/w nicotinamide mononucleotide,
52.47% w/w resveratrol,
0.31% w/w astaxanthin
26.73% w/w berberine.

7. The nutraceutical composition as claimed in claim 1, wherein a specific proportion of the nutraceutical composition comprises:
39.76% w/w nicotinamide mononucleotide,
9.94% w/w pterstobline,
0.60% w/w astaxanthin and
49.70% w/w berberine.

8. The nutraceutical composition as claimed in claim 1, wherein a specific proportion of the nutraceutical composition comprises:
80.00% w/w nicotinamide mononucleotide and
20.00% w/w pterstobline.

9. The nutraceutical composition as claimed in claim 1, wherein a specific proportion of the nutraceutical composition comprises:
39.76% w/w nicotinamide mononucleotide,
9.94% w/w pterstobline,
0.60% w/w astaxanthin and
49.70% w/w metformin.

10. The nutraceutical composition as claimed in claim 1, wherein a specific proportion of the nutraceutical composition comprises:
44.44% w/w nicotinamide mononucleotide and
55.56% w/w metformin.

11. The nutraceutical composition as claimed in claim 1, wherein a specific proportion of the nutraceutical composition comprises:
44.15% w/w nicotinamide mononucleotide,
55.19% w/w metformin and
0.66% w/w astaxanthin.
